(19) United States
(12) Patent Application Publication
(10) Pub. No.: US 2005/0065542 A1
(43) Pub. Date: Mar. 24, 2005

(54) Surgical cutting tools and related methods

(75) Inventor: Paul Mansfield, Houston, TX (US)

Correspondence Address: FULBRIGHT & JAWORSKI L.L.P., 600 CONGRESS AVE., SUITE 2400, AUSTIN, TX, 78701, US

(73) Assignee: Board of Regents, The University of Texas System

(21) Appl. No.: 10/887,159

(22) Filed: Jul. 8, 2004

Related U.S. Application Data
(60) Provisional application No. 60/485,562, filed on Jul. 8, 2003.

(51) Int. Cl. A61B 17/32

(52) U.S. Cl. 606/167

(57) ABSTRACT

Surgical cutting tools, methods of using them, and kits containing them. One of the surgical cutting tools includes a cutting portion and a handle portion that meet at an angle greater than 20 degrees, and the cutting portion includes a sharp bent edge.

### CROSS-REFERENCE(S) TO RELATED APPLICATION(S)

This application claims priority to U.S. Provisional Patent Application Ser. No. 60/485,562, filed Jul. 8, 2003, the entire contents of which are expressly incorporated by reference.

### BACKGROUND OF THE INVENTION

1. Field of the Invention

The present invention relates generally to surgical cutting tools. More specifically, the invention relates to surgical cutting tools configured to excise tissue, such as superficial skin lesions, or other organic matter. The invention also relates to methods for excising tissue from a subject using the same surgical cutting tools, and to kits that include one or more the present surgical cutting tools.

2. Description of Related Art

Various types of surgical cutting tools have been utilized for cutting skin or excising skin lesions. Skin lesions may be flat or protuberant, such as nevi, acrochordons, and warts. Perhaps the most common tool used to excise skin lesions is the scalpel, which is used much like a knife. However, scalpels may be difficult to use when cutting away protuberant lesions on the skin such as moles because the blades are generally stiff. This is particularly true for concave areas. Therefore, the individual using the scalpel has to cut at an incline to the skin surface all the way around the lesion to remove any subsurface portion.

Some medical professionals have found it necessary to take a conventional, thin, flexible steel blade and bend it into an arcuate or U-shape to cut away the protruberant lesion. Since a conventional razor blade is quite thin, the sides of the blade are quite uncomfortable to the user's fingers, and might even cut the fingers. Also, the keen front and rear margins of these blades extend to the sides of the blade which the user grasps, and therefore present an additional danger to the fingers.

### SUMMARY OF THE INVENTION

The present invention provides for surgical cutting tools that can facilitate the excision of tissue and lesions, such as superficial skin lesions and internal tissues such as intra-abdominal tumors. One embodiment is a surgical cutting tool that includes a cutting portion and a handle portion that meet at an angle greater than 20 degrees, where the cutting portion includes a sharp bent edge. A sharp bent edge is a sharp edge that is bent.

Another embodiment is a surgical cutting tool that includes first and second portions that meet at an angle greater than 20 degrees, where the first portion is configured for attachment to a handle, and the second portion is curved and has a cutting edge. The second portion may be characterized by a radius or radii of curvature.

Another embodiment is a surgical cutting tool that includes a handle portion configured for attachment to a non-scissor handle; and an arc-shaped cutting portion that meets the handle portion at an angle greater than 20 degrees, the cutting portion defining an arc-shaped space and having a cutting edge facing away from the arc-shaped space.

A further embodiment is a surgical cutting tool that includes a non-scissor handle portion having an undersurface; and a preformed arc-shaped cutting portion connected to the handle portion, the cutting portion (1) having an undersurface, at least a portion of which is bent back against at least a portion of the undersurface of the non-scissor handle portion; (2) defining an arc-shaped space; and (3) having a cutting edge facing away from the arc-shaped space.

An additional embodiment is a surgical cutting tool that includes a non-scissor handle portion having a surface that faces a subject when the tool is used; and a preformed arc-shaped cutting portion connect to the handle portion, the cutting portion (1) having a surface that faces the subject when the tool is used; (2) defining an arc-shaped space; and (3) having a cutting edge facing away from the arc-shaped space; where at least a portion of the surface of the cutting portion is bent back against at least a portion of the surface of the non-scissor handle portion.

Another embodiment is a method of excising tissue from a subject that includes contacting one of the present surgical cutting tools with tissue from a subject.

Another embodiment is a kit that includes one or more of the present surgical cutting tools.

Other embodiments of the present surgical tools, kits and methods are possible, some of which are described below.

### BRIEF DESCRIPTION OF THE DRAWINGS

The following drawings demonstrate certain aspects of the present methods and devices. They illustrate by way of example and not limitation.

FIGS. 1A-1C show a perspective view (FIG. 1A), front view (FIG. 1B), and side view (FIG. 1C) of one of the present surgical cutting tools.

FIG. 2 shows a side view of one of the present surgical cutting tools.

FIG. 3 shows an enlarged view of the view shown in FIG. 1C.

FIGS. 4A-4B show a view of the undersurface (FIG. 4A) and an enlarged perspective view (FIG. 4B) of the surgical cutting tool shown in FIGS. 1A-1C.

FIGS. 5A-5E show a perspective view of one of the surgical cutting tools (FIG. 5A) that is configured for attachment to a handle (FIG. 5B). A perspective view (FIG. 5C), front view (FIG. 5D), and side view (FIG. 5E) of the surgical cutting tool shown in FIG. 5A attached to the handle depicted in FIG. 5B are shown.

FIGS. 5F-5H show different side views of portions of embodiments of the present surgical cutting tools where the cutting portions have a “V” shape (FIG. 5F), a “W” shape (FIG. 5G), and a shape with two “W”s (FIG. 5H).

FIGS. 6A-6C show a perspective view (FIG. 6A), front view (FIG. 6B), and side view (FIG. 6C) of one of the present surgical cutting tools.

FIG. 7A-7B shows a view of the undersurface (FIG. 7A) and an enlarged perspective view (FIG. 7B) of the surgical cutting tool shown in FIGS. 6A-6C.

FIG. 8 shows an enlarged side view of the surgical cutting tool depicted in FIG. 6C.

FIG. 9 shows a side view of one of the present surgical cutting tools.

FIG. 10 shows a side view of one of the present surgical cutting tools.

FIG. 11 shows one of the present kits.

### DESCRIPTION OF ILLUSTRATIVE EMBODIMENTS

The terms “comprise” (and any form of comprise, such as “comprises” and “comprising”), “have” (and any form of have, such as “has” and “having”), and “include” (and any form of include, such as “includes” and “including”) are open-ended linking verbs. Thus, a surgical cutting tool “comprising” a cutting portion and a handle portion that meet at an angle greater than 20 degrees is a surgical cutting tool that possesses a cutting portion and a handle portion that meet at an angle greater than 20 degrees but is not limited to possessing only these two portions. Likewise, a cutting portion “including” a sharp bent edge possesses a sharp bent edge, but is not excluded from possessing additional features.

The terms “a” and “an” mean one or more than one. The term “another” means at least a second or more.

In some respects, the present surgical cutting tools provide mechanisms that can be used to excise tissue, such as a lesion, with precision such that damage to surrounding healthy tissue is minimized.

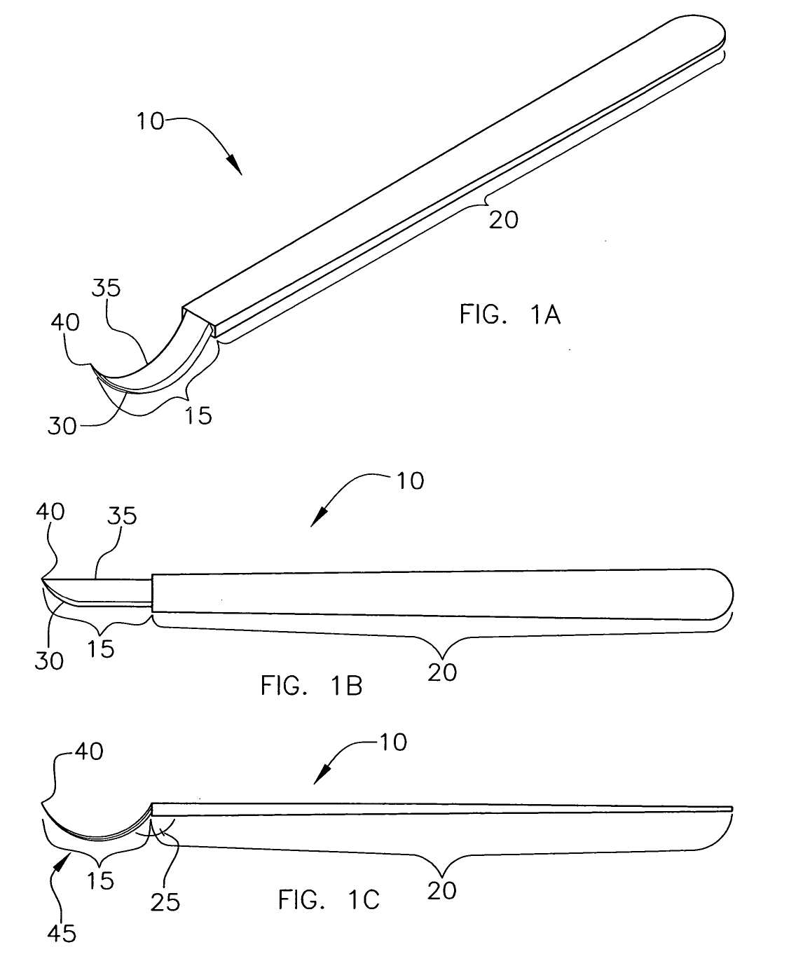

One of the present surgical cutting tools 10 is shown in FIG. 1A-1C. FIG. 1A is a perspective view of surgical cutting tool 10, FIG. 1B is a front view of surgical cutting tool 10, and FIG. 1C is a side view of surgical cutting tool 10.

As shown in FIG. 1, surgical cutting tool 10 includes a cutting portion 15 and a handle portion 20 that, in this embodiment, comprise one coherent element. Cutting portion 15 and handle portion 20 meet at an angle 25 greater than 20 degrees. Any method known to those of skill in the art can be used to measure angle 25. For example, a protractor may be used to measure angle 25.

Any angle greater than 20 degrees can be used. For example, the cutting portion and handle portion may meet at an angle of 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, or 90 degrees, or any increment of a degree derivable from one of these degrees.

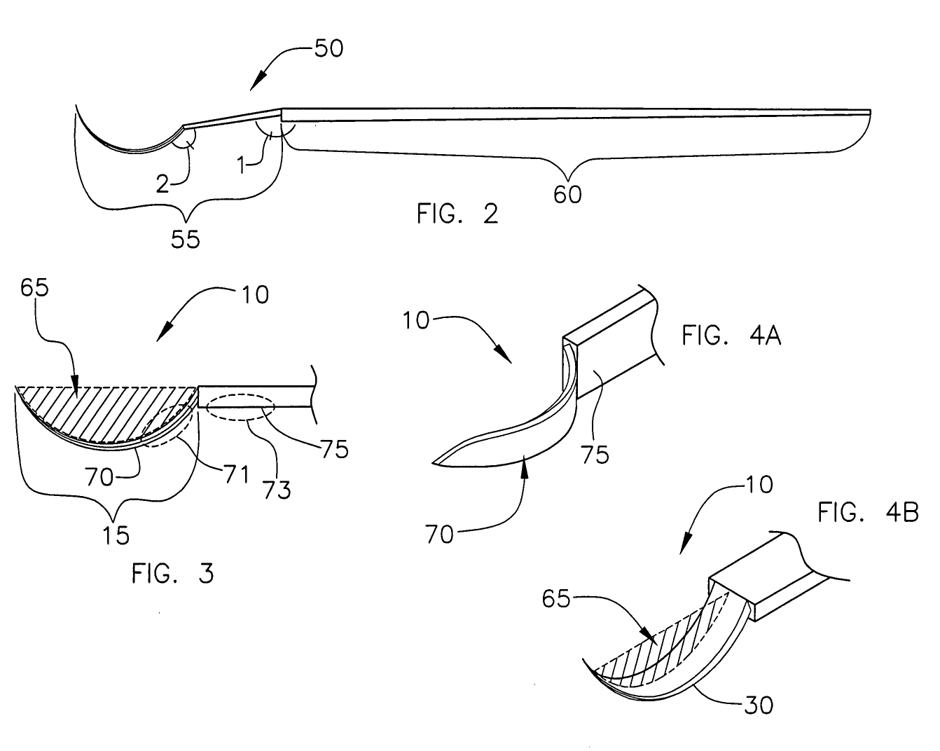

Another of the present surgical cutting tools is surgical cutting tool 50 shown in FIG. 2. Surgical cutting tool 50 includes cutting portion 55, which meets handle portion 60 at angle greater than 20 degrees. In this embodiment, two angles are involved—angles 1 and 2—and their sum exceeds twenty degrees. Where more than one angle is involved, the cutting portion and handle portion of the surgical cutting tool “meet” at an angle greater than 20 degrees provided the sum of those angles exceeds 20 degrees. Thus, incremental angles—such as those shown in FIG. 2—that are less than 20 degrees could satisfy the recited relationship provided the total of all such incremental angles is greater than 20 degrees. Cutting portion 15 includes a sharp bent edge 30 (see FIG. 1A-1C).

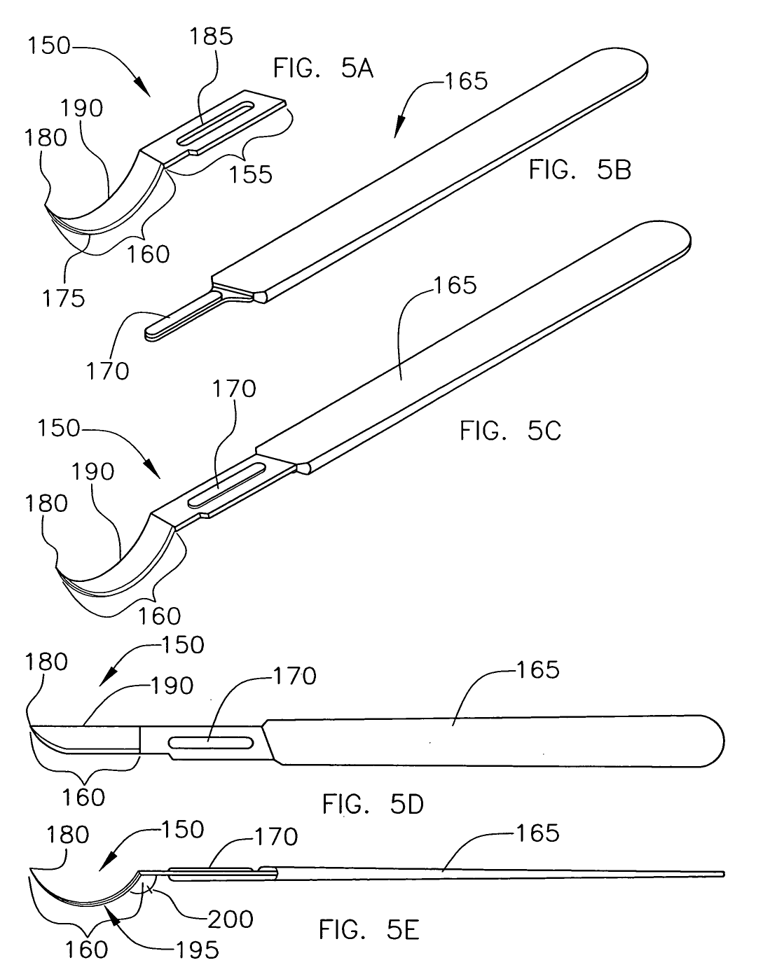

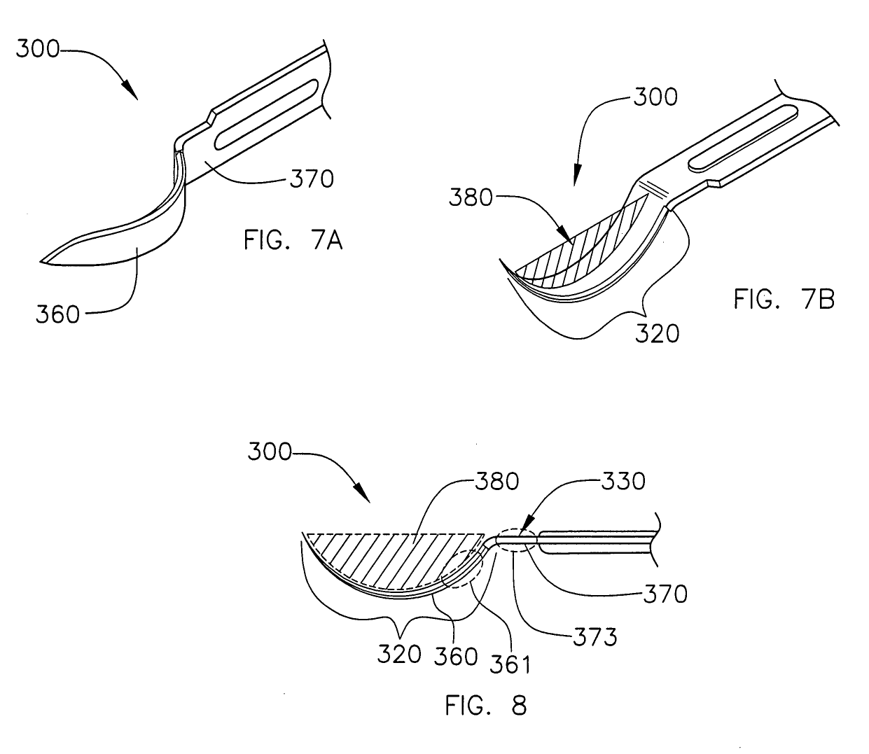

As shown in FIG. 3 and FIGS. 4A-4B, cutting portion 15 of surgical cutting tool 10 may be preformed and arc-shaped. Examples of surgical cutting tools with cutting portions that are not preformed and arc-shaped are disclosed in FIGS. 5-7 of U.S. Pat. No. 5,674,234 (the '234 Patent) at element 20, and FIGS. 3 and 7 of U.S. Pat. No. 4,943,295 (the '295 Patent) at element 11. Instead, those cutting portions, as shown in FIG. 7 of the '234 Patent and FIG. 3 of the '295 Patent, are flat in their resting positions rather than arc-shaped. They must be manipulated to have an arc shape and, therefore, do not possess a preformed arc shape.

FIGS. 3 and 4B shows that cutting portion 15 may also define an arc-shaped space 65. That arc may have one or more radii of curvature, as described in exemplary fashion below. As FIG. 4B shows, sharp bent edge or cutting edge 30 faces away from arc-shaped space 65. The surgical cutting tool shown in U.S. Pat. No. 5,776,154 (the '154 Patent) is an example of a surgical cutting tool that lacks a cutting edge facing away from the arc-shaped space defined by the cutting portion. Instead, the '154 Patent discloses (see FIG. 2) a cutting edge 5 that faces toward the arc-shaped space defined by curved portion 4.

FIGS. 4A and 4B show that cutting portion 30 includes an undersurface 70 that will face a subject in some procedures. FIG. 4A also shows that non-scissor handle portion 20, to which cutting portion 15 is connected, includes undersurface 75, which will also face a subject in some procedures. FIG. 3 shows that at least a portion 71 of undersurface 70 is bent back against at least a portion 73 of undersurface 75. The surgical cutting tools shown in FIGS. 1, 4B and 5B in U.S. Pat. No. 5,292,330 (the '330 Patent) are examples of surgical cutting tools that lack this “bent back” feature. As those figures of the '330 Patent show, no portion of the flatted rear surface 34 of element 18 is bent back against any portion of the undersurface of the handle portion to which it is connected.

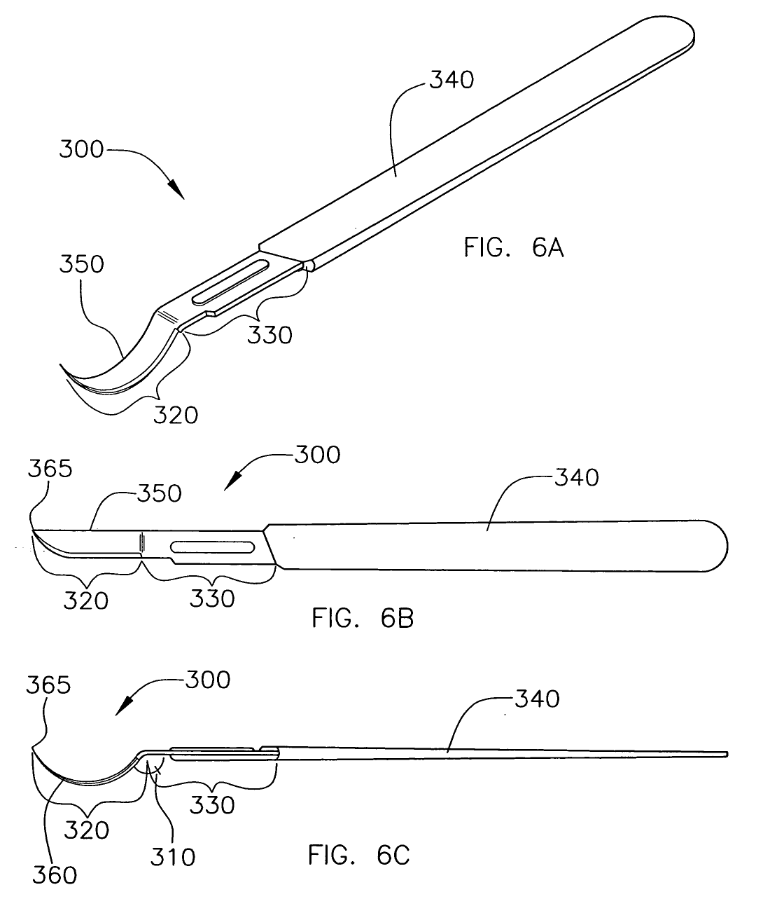

A “non-scissor” handle portion is defined as a handle portion that is not configured to be used as part of a pair of scissors. In contrast, the instrument shown in FIGS. 6A-6C of U.S. Pat. No. 5,478,347 is an example of a surgical cutting tool that includes scissor handle portions 72 and 74.

In certain embodiments, cutting portion 15 may be rigid, semi-rigid, or flexible. One of ordinary skill in the art will be familiar with methods of construction and materials that can be used in the manufacturing process to make cutting portion 15 rigid, semi-rigid, or flexible. For example, the flexibility of cutting portion 15 may be the result of the nature of the material used to construct the cutting portion, or the method of construction of the cutting portion.

In certain embodiments, cutting portion 15 may include more than one sharp edge. For example, in the embodiment of the surgical cutting tool 10 shown in FIG. 1, a second sharp edge may be positioned along the edge 35 that is opposite sharp bent edge 30.

In certain embodiments of the present surgical cutting tools, such as surgical cutting tool 10, the cutting portion, such as cutting portion 15, may be curved or semicircular. In certain embodiments, the cutting portion may have a uniform radius of curvature.

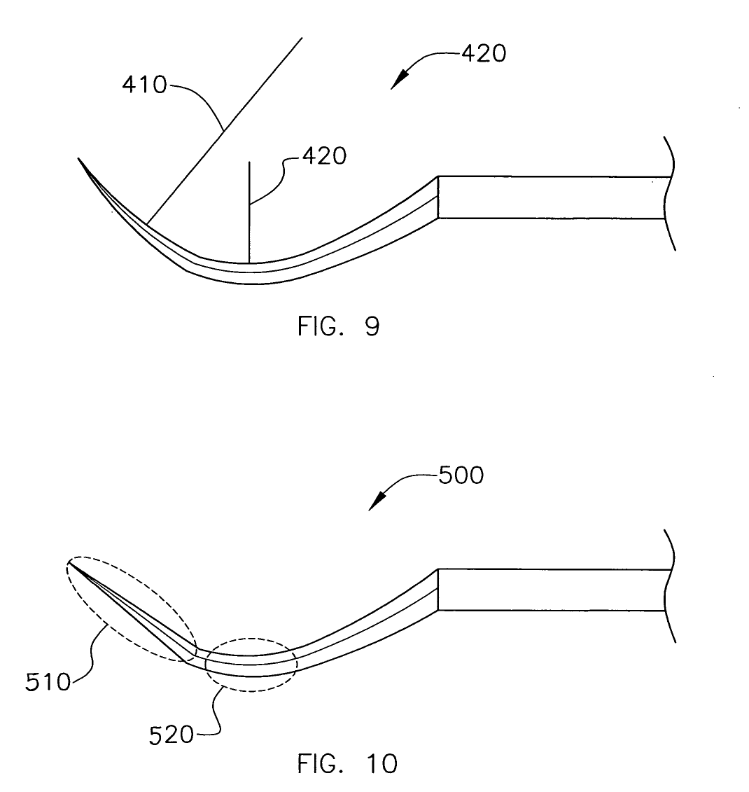

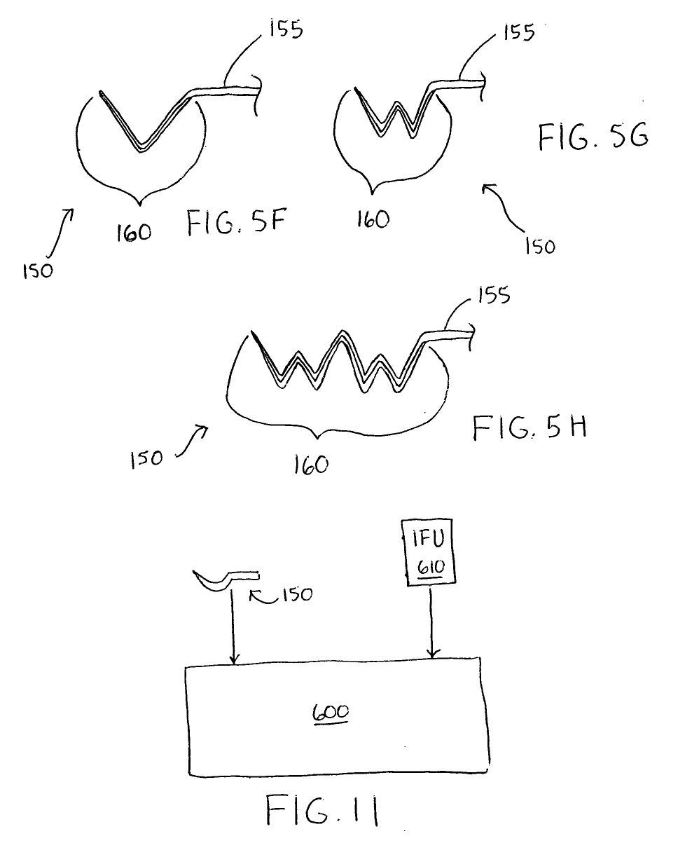

The cutting portion alternatively may have more than one radii of curvature. For example, FIG. 9 depicts one embodiment of the present surgical cutting tools 400 that has a cutting portion with more than one radii of curvature. Surgical cutting tool 400 has a cutting portion with a radius of curvature 420 that is smaller than radius of curvature 410. FIG. 10 demonstrates another embodiment of the present surgical cutting tools 500 that includes a cutting portion with variable radii of curvature. Area 520 of the cutting portion of cutting tool 500 is curved, whereas area 510 is not curved, with a radius of curvature of zero. Thus, some embodiments of the present cutting tools include cutting portions that have more than one radii of curvature, but also include cutting portions having only a segment that is curved.

The radius or radii of curvature of a given cutting portion may be chosen based on the intended application of the device. For example, a device to be used in excising relatively large lesions may have a cutting portion with a larger radius of curvature than one designed for excising smaller lesions. The radius or radii of curvature will depend in part on the length of the cutting portion and, more particularly, on the length of the cutting edge of the cutting portion. In certain embodiments, the radius of curvature of cutting portion 15 of surgical cutting tool 10 is between 8 millimeters and 4 centimeters. In one embodiment, the radius of curvature is 1.5 centimeters.

In the embodiment shown in FIG. 1, surgical cutting tool 10 tapers to form a point 40. In this same embodiment, sharp edge 30 of surgical cutting tool 10 is curved as a result of that taper and the arc shape of cutting portion 15. The cutting portions of the present surgical cutting tools can have any suitable length.

In another embodiment of the present surgical cutting tools shown in FIGS. 5A-5E, surgical cutting tool 150 includes a first portion 155 and a second portion 160. First portion 155 is configured for attachment to a handle such as handle 165, which may be a non-scissor handle. In the embodiment shown in FIG. 5A, that configuration takes the form of first portion 155 having an opening 185 that fits over and works with male portion 170 of handle 165. This configuration is well known to those of ordinary skill in the art.

As shown in FIG. 5E, first portion 155 may have an undersurface 195 that faces a subject when the tool is used.

Second portion 160 of tool 150 is curved and has a cutting edge 175. Second portion 160 meets first portion 155 at a sharp angle 200 (see FIG. 5E) that is greater than 20 degrees. For example, second portion 160 may meet handle portion at an angle of 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, or 90 degrees, or any increment of a degree derivable from one of these degrees. Just as discussed above in reference to the embodiment shown in FIG. 2, the first portion of this embodiment of the present surgical cutting tools may meet the second portion at an angle greater than 20 degrees, where multiple incremental angles of less than 20 degrees are involved.

In some embodiments, second portion 160 defines an arc-shaped space, and cutting edge 175 faces away from that arc-shaped space. This is true of the embodiment shown in FIGS. 5A-5E. The shape of that arc may be characterized by one or more radii of curvature.

In other embodiments, second portion 160 (as well as the cutting portions described elsewhere in this document) may not be arc-shaped; instead, it may be shaped like a “V” (see FIG. 5F), a “W” (see FIG. 5G), two “W”s (see FIG. 5H), or the like, so as to be best suited for the particular type of tissue removal that is needed. In such embodiments, however, at least a portion of the undersurface of those features will be bent back against at least a portion of the undersurface of the first, or non-scissor handle, portion to which they are connected.

Handle 165 can be any handle known to those of ordinary skill in the art. For example, the handle may be a BARD-PARKER™ handle. A surgeon's choice of a handle may depend on the application for which the surgical cutting tool will be used.

Surgical cutting tool 150 may be rigid, semi-rigid, or flexible. In certain embodiments, only first portion 155 is rigid (or semi-rigid or flexible) and in other embodiments, only second portion 160 is rigid (or semi-rigid or flexible). In other embodiments, first portion 155 and second portion 160 are both rigid (or semi-rigid or flexible). The extent of flexibility can vary between the first and second portions, as can it between the cutting and handle portions of other described embodiments.

As shown in FIGS. 5A, 5C and 5D, second portion 160 tapers to a point 180. In other embodiments, second portion 160 may not taper to a point; it may, for example, have a squared-off end, as may the cutting portion of the other embodiments. While second portion 160 is shown as having one cutting edge 175, in other embodiments, second portion 160 may have more than one cutting edge. For example, there may be a second cutting edge along edge 190 that is opposite first cutting edge 175.

FIG. 5C shows surgical cutting tool 150 attached to handle 165. Specifically, first portion 155 of surgical cutting tool 150 is attached to handle 165 in an interlocking fashion (as is well known in the art) such that first portion 155 is firmly affixed to handle 165.

In the embodiment of the present surgical cutting tools shown in FIG. 5, second portion 160 has a uniform radius of curvature. That radius of curvature can have the same range discussed above. In one embodiment, radius of curvature of second portion 160 is 1.5 cm. The radius of curvature will be determined at least in part based on the application for which the surgical cutting tool will be used. Second portion 160 need not have a uniform radius of curvature; any combination of radii of curvature suited for the desired application may be used if a curved second portion is desired.

FIGS. 6A-6C show an embodiment of the present surgical cutting tools similar to surgical cutting tool 150, except that the first and second portions meet at a curve rather than a sharp angle. Surgical cutting tool 300 includes a non-scissor handle portion 330 having an undersurface 370, configured for attachment to a non-scissor handle 340. Surgical cutting tool 300 includes a preformed arc-shaped cutting portion 320 connected to handle portion 330 (in this case, the two portions are connected by virtue of both being part of the same piece of material). Cutting portion 320 has an undersurface 360, at least a portion of which is bent back against at least a portion of undersurface 370 of non-scissor handle portion 330 (see FIGS. 7A and 7B). Cutting portion 320 defines an arc-shaped space 380 (see FIGS. 7B and 8), and has a cutting edge 320 that faces away from arc-shaped space 380.

Undersurface 370, which may also be described as simply a “surface,” of non-scissor handle portion 330 will face a subject when the tool is used for certain procedures. Arc-shaped cutting portion 320 has an undersurface 360, which may also be described as simply a surface, that will face the subject when the tool is used for certain procedures. As shown in FIG. 8, at least a portion 361 of undersurface 360 of cutting portion 320 is bent back against at least a portion 373 of undersurface 370 of non-scissor handle portion 370. Thus, portions of undersurfaces that are “bent back” against each other include those portions that meet each other in a curve (as shown in FIG. 8).

Arc-shaped cutting portion 360 may have a uniform radius of curvature, or multiple radii of curvature. Alternatively, it may have the same alternative shapes (e.g., “V”, “W”, etc.) described above with reference to tool 150. The radius of curvature may be between 4 mm and 8 cm. In one embodiment, the radius of curvature is 1.5 cm. Cutting portion 360 may taper to a point 365, as shown in FIGS. 6A and 6B. Cutting portion 360 may be semicircular, as shown in FIG. 6C. In some embodiments, cutting portion 360 is flexible.

Any method known to those of ordinary skill in the art can be used to construct the present surgical cutting tools. For example, the present surgical cutting tools may be stamped out of metal, shaped using heat, and annealed. The present cutting portions (and first portions) and the present handle portions (and second portions) may be constructed in a single process, or may be constructed separately and then permanently attached in a separate step. For example, in embodiments such as the one in FIG. 1 where the surgical cutting tool includes the handle, cutting portion 15 and handle portion 20 may be permanently attached together using any suitable means of attachment (e.g., adhesive, heat, or the like) following the separate construction of each piece. The surgical cutting tools of the present invention may be disposable (see those in FIGS. 5A-5E and 6A-6C).

Furthermore, the present surgical cutting tools may be packaged and sold in a kit. Referring to FIG. 11, one or more of the present surgical devices—e.g., those with cutting portions and handle portions that comprise one coherent element (such as the embodiment shown in FIG. 1A) or those that are configured for attachment to a handle after they are unpackaged (such as the embodiment shown in FIG. 5A)—may be sterilized (as may any of the present surgical cutting tools using known techniques in preparation for use in a procedure on a patient) and placed in a surgical cutting tool-retaining structure 600. Structure 600 may be sealed to maintain the sterility of the one or more surgical devices, and may take the form of a transparent synthetic pouch, a tray that is configured to securely hold one or more surgical cutting tools, or any other structure suitable for holding one or more of the present surgical devices, such as a box, tube, or the like. A protective guard, such as a plastic sheath (not shown), may be placed over the relevant portion of the surgical cutting tool or tools to cover the cutting edge. Optionally, instructions for use (IFU) 610 may be packaged with the surgical cutting tools or tools in structure 600, which may include one or more labels attached to an exterior surface that explain the contents of the kit.

Surgical cutting tools of the present invention can be composed of any material known to those of ordinary skill in the art. For example, the surgical cutting tool can be composed of surgical-grade stainless steel, or any suitable alloy known to those of ordinary skill in the art. In some embodiments, the cutting portion (or first portion) of the surgical cutting tools, the handle portion (or second portion), or both may be composed of a shape-memory material such as a nickel-titanium alloy like nitinol. In other embodiments, the cutting and handle portions are composed of different materials, such as different metals or different alloys. One of ordinary skill in the art would be familiar with the wide range of materials available for use in the construction of the present surgical cutting tools.

The present surgical cutting tools may be advantageously used in procedures that require tissue removal. For example, one of the present surgical cutting tools can be used to excise an elevated skin lesion, such as a skin cancer. In other embodiments, the present surgical cutting tools can be used to harvest a small area of skin for use as a skin graft. In still other embodiments, the present surgical cutting tools can be used to excise intra-abdominal lesions such as those on the liver, pancreas, peritoneum, etc. Still other embodiments of the present surgical cutting tools can be used to excise surface lesions of internal organs such as the pancreas or liver, where the conventional process involves excising the lesion in a short stabbing motion and, by necessity, the tip of the conventional instrument goes further into the tissue than what is excised and can—as a result—injure a deeper structure such as the pancreatic duct, etc. One of ordinary skill in the art would be familiar with the wide range of procedures that may be accomplished using the present surgical cutting tools.

The present surgical cutting tools can be made and used without undue experimentation in light of this disclosure. The present surgical cutting tools need not be made in the exact disclosed forms, or combined in the exact disclosed configurations to fall within the scope of the claims and their equivalents. Instead, it is possible to make substitutions, modifications, additions and/or rearrangements of the disclosed features without deviating from the scope of the present surgical cutting tools, which is defined by the claims and their equivalents. Further, although the present methods can be practiced using the specific techniques disclosed above, such methods can also be practiced using other techniques.

The appended claims are not to be interpreted as including means-plus-function limitations, unless such as limitation is explicitly recited in a given claim using the phrase(s) “means for” and/or “step for,” respectively.

1. A surgical cutting tool comprising:
a cutting portion and a handle portion that meet at an angle greater than 20 degrees, the cutting portion including a sharp bent edge.

2. The surgical cutting tool of claim 1, where at least the cutting portion is sterilized.

3. The surgical cutting tool of claim 1, where the cutting portion is flexible.

4. The surgical cutting tool of claim 1, where the cutting portion is curved.

5. The surgical cutting tool of claim 4, where the cutting portion has a uniform radius of curvature.

6. The surgical cutting tool of claim 5, where the uniform radius of curvature is between 4 millimeters and 8 centimeters.

7. The surgical cutting tool of claim 4, where the cutting portion has multiple radii of curvature.

8. The surgical cutting tool of claim 1, where the cutting portion tapers to a point.

9. The surgical cutting tool of claim 1, where the cutting portion is semicircular.

10. A surgical cutting tool comprising:
first and second portions that meet at an angle greater than 20 degrees;
where the first portion is configured for attachment to a handle, and the second portion is curved and has a cutting edge.

11. The surgical cutting tool of claim 10, where at least the second portion is sterilized.

12. The surgical cutting tool of claim 10, where the second portion tapers to a point.

13. The surgical cutting tool of claim 12, where the second portion has two cutting edges.

14. The surgical cutting tool of claim 10, where the second portion is flexible.

15. The surgical cutting tool of claim 14, where the first and second portions are flexible.

16. The surgical cutting tool of claim 10, where the first and second portions meet an a sharp angle.

17. The surgical cutting tool of claim 10, where the second portion has a uniform radius of curvature.

18. The surgical cutting tool of claim 10, where the second portion comprises a radius of curvature of between 4 millimeters and 8 centimeters.

19. The surgical cutting tool of claim 10, where the cutting portion has multiple radii of curvature.

20. A surgical cutting tool comprising:
(a) a handle portion configured for attachment to a non-scissor handle; and
(b) an arc-shaped cutting portion that meets the handle portion at an angle greater than 20 degrees, the cutting portion defining an arc-shaped space and having a cutting edge facing away from the arc-shaped space.

21. The surgical cutting tool of claim 20, where at least the cutting portion is sterilized.

22. The surgical cutting tool of claim 20, where the cutting portion has a uniform radius of curvature.

23. The surgical cutting tool of claim 20, where the cutting portion has multiple radii of curvature.

24. The surgical cutting tool of claim 20, where the cutting portion is flexible.

25. The surgical cutting tool of claim 22, where the uniform radius of curvature is between 4 millimeters and 8 centimeters.

26. The surgical cutting tool of claim 20, where the cutting portion tapers to a point.

27. The surgical cutting tool of claim 20, where the cutting and handle portions meet at a sharp angle.

28. A surgical cutting tool comprising:
(a) a non-scissor handle portion having an undersurface; and
(b) a preformed arc-shaped cutting portion connected to the handle portion, the cutting portion:
(i) having an undersurface, at least a portion of which is bent back against at least a portion of the undersurface of the non-scissor handle portion;
(ii) defining an arc-shaped space; and
(iii) having a cutting edge facing away from the arc-shaped space.

29. The surgical cutting tool of claim 28, where at least the cutting portion is sterilized.

30. The surgical cutting tool of claim 28, where the cutting portion has a uniform radius of curvature.

31. The surgical cutting tool of claim 28, where the cutting portion has multiple radii of curvature.

32. The surgical cutting tool of claim 28, where the cutting portion is flexible.

33. The surgical cutting tool of claim 30, where the uniform radius of curvature is between 4 millimeters and 8 centimeters.

34. The surgical cutting tool of claim 28, where the cutting portion tapers to a point.

35. The surgical cutting tool of claim 28, where the cutting and handle portions meet at a sharp angle.

36. The surgical cutting tool of claim 28, where the cutting and handle portions meet at a curve.

37. A surgical cutting tool comprising:
(a) a non-scissor handle portion having a surface that faces a subject when the tool is used; and
(b) a preformed arc-shaped cutting portion connected to the handle portion, the cutting portion:
(i) having a surface that faces the subject when the tool is used;
(ii) defining an arc-shaped space; and
(iii) having a cutting edge facing away from the arc-shaped space;
(c) where at least a portion of the surface of the cutting portion is bent back against at least a portion of the surface of the non-scissor handle portion.

38. The surgical cutting tool of claim 37, where at least the cutting portion is sterilized.

39. The surgical cutting tool of claim 37, where the cutting portion has a uniform radius of curvature.

40. The surgical cutting tool of claim 37, where the cutting portion has multiple radii of curvature.

41. The surgical cutting tool of claim 37, where the cutting portion is flexible.

42. The surgical cutting tool of claim 39, where the uniform radius of curvature is between 4 millimeters and 8 centimeters.

43. The surgical cutting tool of claim 37, where the cutting portion tapers to a point.

44. The surgical cutting tool of claim 37, where the cutting and handle portions meet at a sharp angle.

45. The surgical cutting tool of claim 37, where the cutting and handle portions meet at a curve.

46. (Canceled)

47. (Canceled)
